(19) United States
(12) Patent Application Publication
(10) Pub. No.: US 2011/0152802 A1
(43) Pub. Date: Jun. 23, 2011

(54) Incontinence diaper/underwear system

(76) Inventors: Lorenzo DiCamillo, Staten Island, NY (US); Fred Carpentieri, Staten Island, NY (US)

(21) Appl. No.: 12/927,740

(22) Filed: Nov. 23, 2010

Related U.S. Application Data
(60) Provisional application No. 61/283,186, filed on Nov. 30, 2009.

(51) Int. Cl. A61F 5/453; A61F 5/455

(52) U.S. Cl. 604/349; 604/347

(57) ABSTRACT

The Incontinence Diaper/Underwear System is a wearable undergarment, that is specially configured with a urination receptacle in the man's version. The Incontinence Diaper/Underwear System is essentially envisioned as a cotton or rubber brief-type undergarment that features a standard, elastic waistband that expands to accommodate waist sizes from twenty-seven inches to forty-six inches (27″-46″). The Incontinence Diaper/Underwear System is equipped with a handy plastic pouch unit, which serves to house urine should the need arise. This is accomplished via a rubber hose attachment that connects the pouch with a comfortable, absorbent “collar” in the crotch of the undergarment, which when worn house the user's penis and testicles. In addition, a handy drain valve is positioned at the bottom of this receptacle to facilitate emptying.

### CLAIM OF PRIORITY

This patent application claims priority under 35 USC 119(e)(1) from U.S. Provisional Patent Application Ser. No. 61/283,186 filed Nov. 30, 2009, of common inventorship herewith entitled, “Incontinence Diaper/Underwear System.”

### FIELD OF THE INVENTION

The present invention pertains to the field of incontinence devices and more specifically to the field of diapers and diaper systems.

### BACKGROUND OF THE INVENTION

The prior art has put forth several designs for devices for incontinence. Among these are:

U.S. Pat. No. 5,948,910 to Joseph J. Berke describes a urinary incontinence device and method which describes an undergarment worn closely to the body by males and includes an opening or hole through which the penis passes.

U.S. Pat. No. 6,419,665 to Morton H. Cohen describes a men's undergarment for incontinence comprising a pair of shorts having a pocket portion in the crotch of the shorts for receiving the protrusion of the penis.

U.S. Pat. No. 7,090,666 to Mark Miskie describes a moisture management incontinence device comprising a pad having a moisture impervious covering and wraps around the penis.

None of these prior art references describe the present invention.

### SUMMARY OF THE INVENTION

It is an object of the invention to provide an improved diaper system for use by incontinent men, women and children. Male version and female versions are available for both adults and children.

### BRIEF DESCRIPTION OF THE DRAWINGS

FIG. 1 is a front perspective view of the garment of the present invention configured for use by males.

FIG. 2 is a front perspective view of the garment of the present invention configured for use by females.

FIG. 3 is a schematic diagram of the urine reservoir system of the present invention. A solid plastic wire or strip is inside the flow tube to prevent kinking or bending.

FIG. 4 is a front perspective view of the garment of the present invention being worn by a male.

FIG. 5 is a front perspective view of the garment of the present being worn by a female or child (not shown).

FIG. 6 shows a schematic overall view of the incontinence system of the present invention.

FIG. 7 shows the organ entrance membrane opening of the incontinence system of the present invention.

FIG. 8 is a schematic side view of the incontinence system of the present invention.

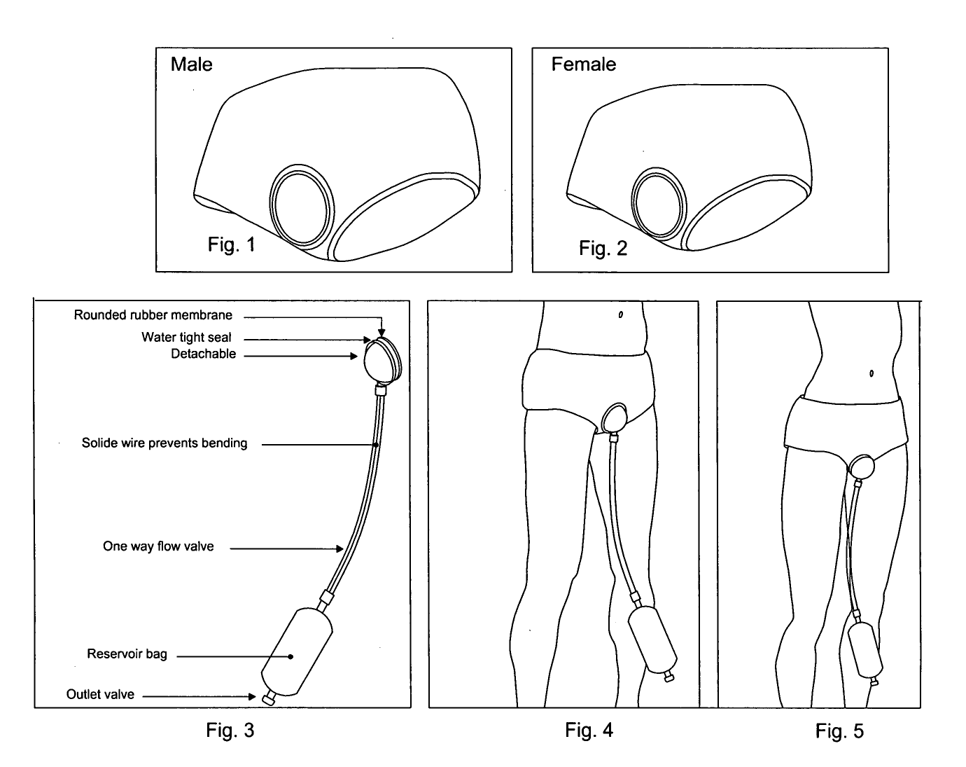

### DETAILED DESCRIPTION OF THE INVENTION

Incontinence affects many people and is a public health problem of enormous proportions. This condition is one of the most common reasons why people enter nursing homes, where weary caregivers crumble under the strain of constantly changing diapers and cleaning wet sheets. There are, of course, varying degrees of incontinence. Some people have occasional leaks when they cough, sneeze, or exercise. Others, however, have little or no control of their urine, and this affects virtually every aspect of their existence.

While incontinence mostly affects older adults, it is not confined to them. It is seen in people of all ages, including children, although the incidence rises with each passing decade. Although it is widely known that women are twice as likely to develop the condition as men, a significant percentage of the male population has to deal with incontinence on a daily basis. Prostate cancer surgery is a major reason men become incontinent. In addition, diabetes, neurologic disease, and spinal cord injury also can interfere with bladder control in males.

While a physical ailment, perhaps one of the most distressing side effects of incontinence is psychological. Particularly, the lack of urinary control invariably results in the staining of undergarments and outer clothing, which is very embarrassing and demeaning to sufferers. In addition to humiliation, incontinence can also ruin garments, resulting in costly dry cleaning charges and even clothing replacement. To help protect their clothing, as well as their self-confidence, patients with these conditions turn to absorbent, disposable diaper-like garments and underwear liners to serve as a shield between their clothing and the source of any seepage.

While adult diapers can be very effective in achieving their intended purpose, these units tend to be extremely bulky and uncomfortable to wear. In addition, the vast majority of undergarment protectors currently available on the market are geared toward women, and designed to accommodate their physical form. As such, men who suffer from uncontrolled discharge have very few options to help combat their condition.

The present invention, hereinafter referred to as the Incontinence Diaper/Underwear System, is a clever invention that offers people a practical assist in incontinence and similar medical problems.

Simply stated, the Incontinence Diaper/Underwear System is a wearable undergarment, that is specially configured with a urination receptacle in the man's version. The Incontinence Diaper/Underwear System is essentially envisioned as a cotton or rubber brief-type undergarment that features a standard, elastic waistband that expands to accommodate waist sizes from twenty-seven inches to forty-six inches (27″-46″). The Incontinence Diaper/Underwear System is equipped with a handy plastic pouch unit, which serves to house urine should the need arise. This is accomplished via a rubber hose attachment that connects the pouch with a comfortable, absorbent “collar” in the crotch of the undergarment, which when worn house the user's penis and testicles. In addition, a handy drain valve is positioned at the bottom of this receptacle to facilitate emptying.

Use of the Incontinence Diaper/Underwear System is very simple and straightforward. First, the user removes an undergarment from its sterile packaging, and follows package instructions for donning the unit. Next, the user simply inserts the penis into the collar that connects the hose to the urine housing pouch. After completing the remainder of the dressing regimen, the user goes about any desired activity, confident in the supreme protection that the Incontinence Diaper/Underwear System affords. After use, the pouch is easily removed and the contents properly disposed of, and the unit can be washed, disinfected, dried, and put away until needed again.

This device can be used for pediatrics, male or female. This is a green product invention as it is safe for the environment because it reduces waste unlike diapers sold on the market today. This device allows urine to collect into a container/reservoir such that the contents of said container/reservoir can be emptied.

This device eliminates the waste of having to frequently change diaper/underwear every time urination occurs. The diaper would require changing only after a bowel movement. This also saves on cost of diapers, as a result of less frequent changes.

This device makes it easier for a caretaker to apply a catheter to a patient. It eliminates standard methods of applying a rubber condom catheter. A rubber condom catheter is uncomfortable, irritating to the penis and skin and restricts circulation. The incontinence device of the present invention is comfortable for either male or female, adults or children.

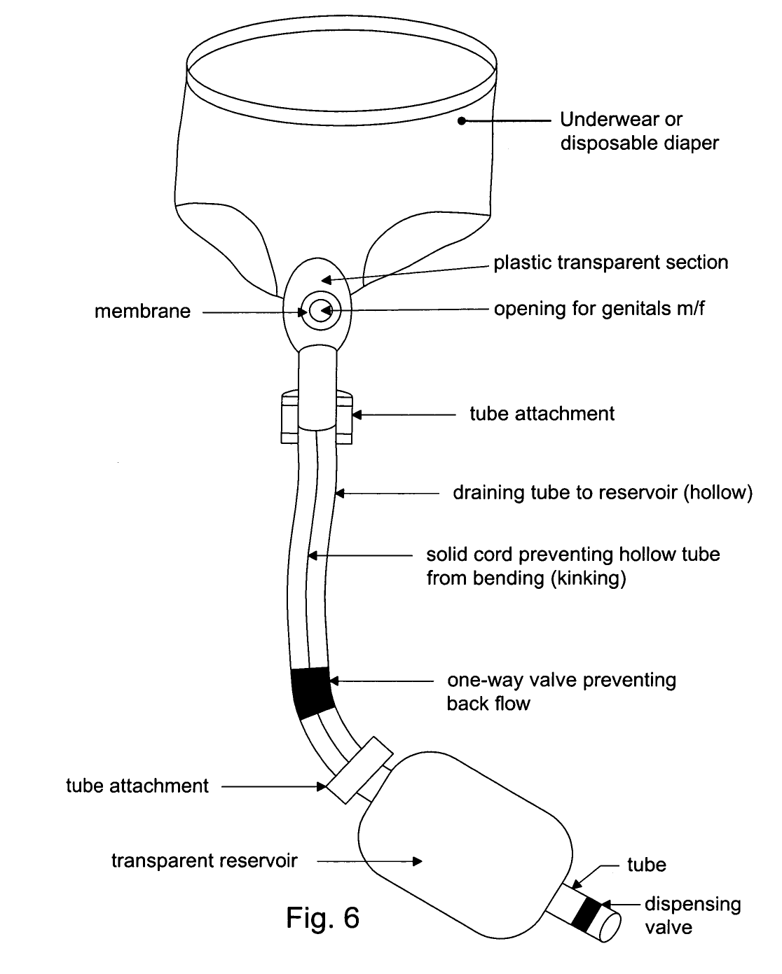

The female version incorporates a soft pressed fit on exterior of genital area. The male version comprises a soft membrane opening that the penis passes through comfortably and leak free. (see FIG. 6)

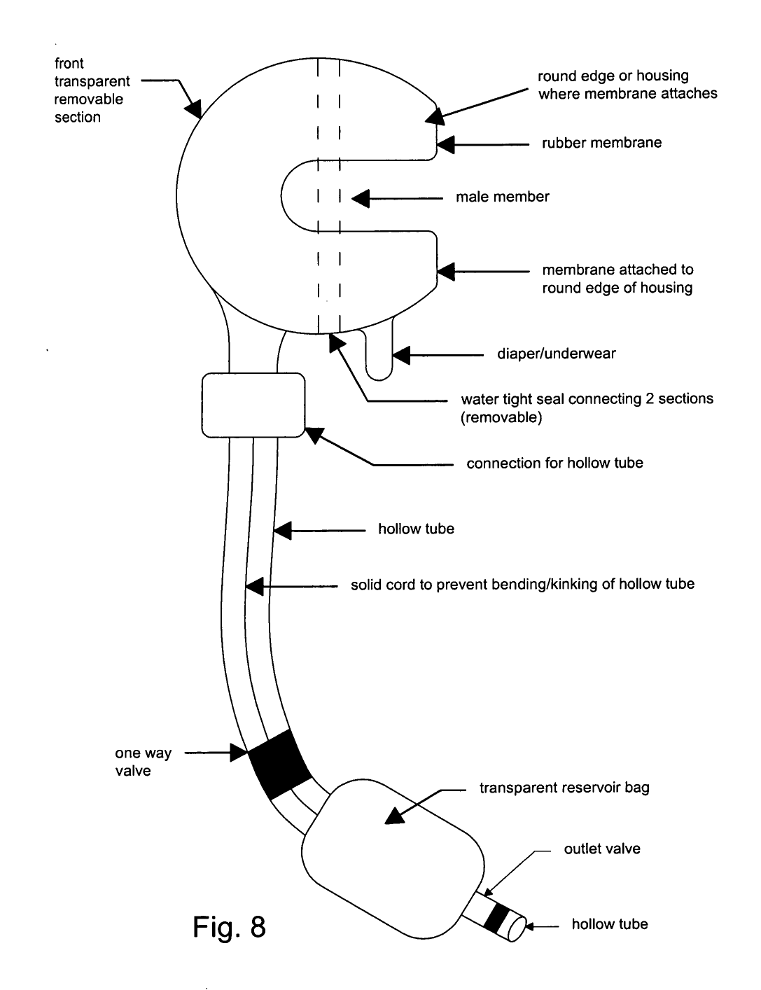

The front section is transparent and removable for application and also for inspection by the nurse or caretaker. (See FIG. 8)

The egg-shaped design, having no corners, allows for extra comfort and lessens the appearance when user is clothed, for non-bed patients/users. (see FIGS. 6, 7, 8)

The housing is designed in two sections for easy attachment and access to genitals, for the male and female versions, and for easy removal of the system without having to remove the diaper/underwear. If used with a disposable diaper, the diaper can be discarded without discarding the rest of the system. (see FIG. 8)

The two sections attach with a leak proof seal with the minimal edge on the undergarment side. The undergarment side or section provides a provides a membrane and an opening.

The membrane attaches to the housing with a round comfortable edge. (see FIG. 8)

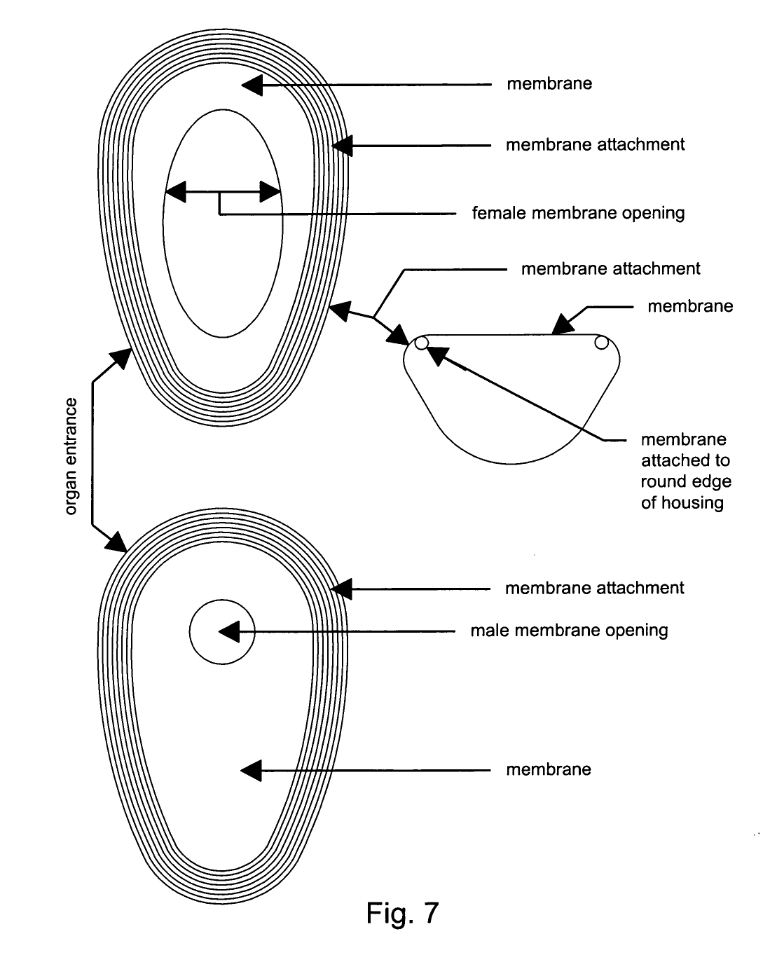

The membrane provides a seal where the skin attaches to the device. The female version housing provides a seal around the appropriately sized opening where the housing is pressed to the external of the genitals. The male version provides a membrane opening to be penetrated by the penis to provide a comfortable leak proof seal. The opening would be larger for a female, and small for a male. (see FIG. 7)

Regarding the underwear/diaper, persons suffering from incontinence for urine and feces may use disposable under garments. Others who are healthy can use non-disposable undergarments. (see FIG. 6).

The transparent removable section empties into the hollow/non-kinking tube, which tube has a one way valve to prevent backflow. (see FIG. 6).

The tube empties into the reservoir, which is a flat, round bag that can be worn under clothing or hung under a bed. The bag is to be transparent so caretaker or nurse can inspect the color of the urine and amount of urine. The reservoir bag will have an outlet tube with a valve for emptying the contents without the need of opening or removing the system. (see FIG. 6).

Unlike other products, this incontinence device separates urine from feces therefore minimizing waste associated with standard disposable diapers/underwear. This system allows for a child/adult, sick/healthy to urinate freely without leakage. Urine collects in a reservoir and can be dispensed at a later time. The system can be removed, reservoir emptied and used again without the need to discard the diaper, thereby eliminating the need for frequent changes.

A person not suffering from incontinence can use the product for work related reasons where there is no access to toilet or if removing special clothing will be of difficulty.

This device can be considered a green product since it saves on the amount of paper material used. Eliminating frequent changes (pediatrics or geriatrics) keep changes to a minimum.

This device is different than other incontinence devices on the market today because the standard condom type catheter applied to the penis irritates the skin, is uncomfortable and reduces circulation causing swelling. A patient would be very uncomfortable, either adult or child. This invention is easy to apply to a child, sick patient or healthy person. A healthy person using it for work related reasons would not be able to use a standard catheter system available today. This invention can be used comfortably.

There are several significant benefits and advantages associated with this clever product invention. Foremost, the Incontinence Diaper/Underwear System provides males who are afflicted with incontinence a simple and effective means of relieving themselves as well as protection. A wearable unit with components that are specifically configured fit the male genitalia; this practical unit offers males a comfortable and secure fit, eliminating the need to rely on products that are designed more for women. In this manner, men who suffer from incontinence and similar conditions are able to go about their daily routines with confidence, ensured that their Incontinence Diaper/Underwear System will allow them to avoid the embarrassment of walking around in visibly stained clothing.

In addition to preventing humiliation, the Incontinence Diaper/Underwear System provides an immediate, private receptacle for urination, offering instant relief when a restroom is not readily available. Whether one is stuck in the middle of a business meeting, enjoying a competition in a sports arena, or in the middle of a motion picture at a movie theater, this product ensures comfortable relief regardless of circumstance. Comfortably worn, the Incontinence Diaper/Underwear System eliminates the need to wear bulky, uncomfortable standard adult diapers, which can also be challenging to operate when it is necessary to utilize the restroom. While this product was primarily conceived for household consumers, doctors and hospitals will surely embrace the Incontinence Diaper/Underwear System as a helpful tool to recommend to patients and to utilize for in-patient care, possibly eliminating the need to rely on an invasive catheter.

An innovative invention, the Incontinence Diaper/Underwear System readily enhances the daily lives of millions of men. Secure and comfortable, this clever product offers a discrete, effective, and easy solution for incontinence and similar problems. Affordably priced, the Incontinence Diaper/Underwear System will be well received by all men who suffer from these conditions.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A diaper system for adult males, comprising a wearable undergarment in the form of a cotton or rubber brief comprising an elastic waistband and a urination receptacle in the form of an egg shaped plastic pouch unit; wherein the pouch connects to a rubber hose attachment and the hose attachment connects with a comfortable, absorbent “collar” in the crotch of the undergarment, which when worn houses the user's penis and testicles.

2. The diaper system of claim 1 further comprising a drain valve positioned at the bottom of the urination receptacle to facilitate emptying.

3. A diaper system for adult female comprising a soft pressed fit on exterior of genital area.

4. A diaper system comprising a transparent front section for inspection by the nurse or caretaker and the front section is removable.

5. A diaper system sized to accommodate male children, comprising a wearable undergarment in the form of a cotton or rubber brief comprising an elastic waistband and a urination receptacle in the form of an egg shaped plastic pouch unit; wherein the pouch connects to a rubber hose attachment and the hose attachment connects with a comfortable, absorbent “collar” in the crotch of the undergarment, which when worn houses the user's penis and testicles.
